(19) United States
(12) Patent Application Publication
(10) Pub. No.: US 2026/0085037 A1
(43) Pub. Date: Mar. 26, 2026

(54) ALKYL ETHER SULFATE SURFACTANTS AND ASSOCIATED METHODS OF MANUFACTURE

(71) Applicant: Dow Global Technologies LLC, Midland, MI (US)

(72) Inventors: Sung-Yu Ku, Manvel, TX (US); Tanner Mcdaniel, Midland, MI (US); Wanglin Yu, Pearland, TX (US); Timothy J. Young, Bay City, MI (US); Wen-Sheng Lee, Midland, MI (US)

(21) Appl. No.: 19/112,609

(22) PCT Filed: Sep. 19, 2023
(86) PCT No.: PCT/US2023/074519

§ 371 Date: Mar. 18, 2025

Related U.S. Application Data
(60) Provisional application No. 63/408,123, filed on Sep. 20, 2022.

(51) Int. Cl. C07C 303/24

(52) U.S. Cl. CPC C07C 303/24

(57) ABSTRACT

A process includes the steps of contacting an olefin, an alcohol and a metallosilicate catalyst to form oligomers of an alcohol ethoxylate having structure (I), wherein R₁ is an alkyl, R₂ is an alkyl, and n has a value of 1 to 3; and sulfating the oligomers of Structure (I) to form oligomers of Structure (II), wherein R₁ is an alkyl, R₂ is selected from the group consisting of an alkyl group, M is selected from the group consisting of a proton, an ammonium cation, a metal cation, a nitrogen cation, a boron cation, a phosphorus cation, triethylamine, triethanolamine, monoethanolamine and combinations thereof, and n has a value of 1 to 3, and wherein 95 mol % or greater of the oligomers of Structure (II) have an n of 1 and 5 mol % or less of the oligomers of Structure (II) have an n of 2 or greater.

### BACKGROUND

### Field of the Invention

The present disclosure generally relates to surfactants and more specifically to alkyl ether sulfate surfactants and associated methods of manufacture.

### INTRODUCTION

Regulators have been increasing restrictions on the amount of 1,4 dioxane (“dioxane”) that may be present in consumer products. For example. New York State has banned all but trace amounts of dioxane in cosmetics, personal care, and cleaning products. Typically, a consumer product must comprise less than 10 parts per million (“ppm”) of dioxane to be compliant with the regulations. One contributor of dioxane in consumer products can be alkyl ethoxy sulfate (“AES”) anionic surfactants.

Dioxane formation from AES surfactants is believed to occur at multiple points in time. A first point of dioxane generation in AES surfactants is during the sulfation process of alcohol ethoxylates to make alcohol ethoxy sulfates. The alcohol ethoxylate intermediates for the production of alcohol ethoxy sulfate surfactants are made via ethoxylation (i.e., the reaction of an alcohol with ethylene oxide) that typically results in a distribution of alcohol ethoxylate oligomers. It is believed that under the sulfation process conditions dioxane can form.

A second point of dioxane generation in AES surfactants is during handling and processing. Handling and processing of sulfated surfactants often involves acidic conditions at ambient temperature or elevated temperatures. Prolonged exposure to acidic environments for the AES surfactants and their alcohol ethoxylate precursors can result in the formation of dioxane. Further, exposure to elevated temperatures during processing (e.g., 280° C.), storage and handling can result in the decomposition of AES surfactants resulting in the formation of dioxane. Therefore, AES surfactants that exhibit a thermal stability would decrease dioxane concentrations in products.

Traditionally, dioxane content in sulfated surfactants and products incorporating the surfactants has been addressed by the use of stripping techniques. For example, where dioxane concentrations were above a target threshold, a stripping process is employed to remove excess dioxane from the sulfated surfactants or product incorporating the surfactant. The stripping process are expensive and time consuming. Further, as dioxane may form over time as a response to how the surfactant or product is handled and further processed, any previously applied stripping techniques may be nullified by the generation of new dioxane. As such, ensuring that a product made from sulfated surfactants is compliant with the appropriate regulations by the time it is sold to an end consumer is a difficult challenge.

In view of the foregoing, it would be surprising and advantageous to discover an alcohol ethoxylate and sulfated surfactant that resists forming 9 ppm or more of dioxane after both sulfation and exposure to elevated temperatures up to 280° C.

### SUMMARY

The inventors of the present application have discovered an alcohol ethoxylate and sulfated surfactant that resists forming 9 ppm or more of dioxane after both sulfation and exposure to elevated temperatures up to 280° C.

The inventors of the present disclosure have discovered that the above-noted properties can be achieved by surfactant compositions where 95 mole percent (“mol %”) or greater of the surfactant has a single ethylene-oxide unit and 5 mol % or less of the surfactant as an ethylene oxide content of 2 or greater. Without being bound by theory, it is believed that the distribution of oligomers created during alkoxylation is responsible for the formation of dioxane in both the alcohol ethoxylates and the sulfated surfactant. It is believed that oligomers with an ethylene oxide (“EO”) number of 2 or greater can undergo a “back-biting” reaction to generate the unwanted dioxane. Surprisingly, by restricting the presence of 2EO unit oligomers to 5 mol % or less, the surfactant is able to resist generating 9 ppm of dioxane both during sulfation and when exposed to 280° C. Further, it has been discovered that contacting an alcohol and an olefin in the presence of a metallosilicate catalyst can produce the above-noted alcohol ethoxylate.

The present invention is particularly useful in the formation of AES surfactants and products made therefrom.

According to a first feature of the present disclosure, a process includes the steps of contacting an olefin, an alcohol and a metallosilicate catalyst to form oligomers of an alcohol ethoxylate having structure (I), wherein R₁ is an alkyl, R₂ is an alkyl, and n has a value of 1 to 3; and sulfating the oligomers of Structure (I) to form oligomers of Structure (II), wherein R₁ is an alkyl, R₂ is selected from the group consisting of an alkyl group, M is selected from the group consisting of a proton, an ammonium cation, a metal cation, a nitrogen cation, a boron cation, a phosphorus cation, triethylamine, triethanolamine, monoethanolamine and combinations thereof, and n has a value of 1 to 3, and wherein 95 mol % or greater of the oligomers of Structure (II) have an n of 1 and 5 mol % or less of the oligomers of Structure (II) have an n of 2 or greater.

According to a second feature of the present disclosure, the olefin is a C₈ to C₁₈ alpha olefin and the alcohol is monoethylene glycol.

According to a third feature of the present disclosure, a sum of carbon atoms in R₁ and R₂ is from 7 to 17.

According to a fourth feature of the present disclosure, a sum of carbon atoms in R₁ and R₂ is from 11 to 13.

According to a fifth feature of the present disclosure, the catalyst to make Structure (I) has a surface area of 680 m²/g as measured according to ASTM D4365-19.

According to a sixth feature of the present disclosure, 98 mol % or greater of the oligomers of Structure (I) have an n of 1 and 2 mol % or less of the oligomers of Structure (I) have an n of 2 or greater.

According to a seventh feature of the present disclosure, a surfactant composition includes oligomers of a surfactant having structure (II), wherein R₁ is an alkyl, R₂ is an alkyl, M is selected from the group consisting of a proton, an ammonium cation, a metal cation, a nitrogen cation, a boron cation, a phosphorus cation, triethylamine, triethanolamine, monoethanolamine and combinations thereof, and n has a value of 1 to 3, and wherein 95 mol % or greater of the oligomers of Structure (II) in the composition have an n of 1 and 5 mol % or less of the oligomers of Structure (II) in the composition have an n of 2 or greater.

According to an eighth feature of the present disclosure, R₁ of Structure (II) is a C₁ to C₁₆ alkyl, R₂ is selected from the group consisting of a C₁ to C₁₆ alkyl, further wherein a sum of carbon atoms in R₁ and R₂ is from 7 to 17.

According to a ninth feature of the present disclosure, 98 mol % or greater of the oligomers of Structure (II) in the surfactant composition have an n of 1 and 2 mol % or less of the oligomers of Structure (II) in the surfactant composition have an n of 2 or greater.

According to a tenth feature of the present disclosure, a sum of carbon atoms in R₁ and R₂ is from 11 to 13.

According to an eleventh feature of the present disclosure, a material includes 0.1 wt % to 99.9 wt % of the surfactant composition.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standards.

IUPAC codes describing Crystal structures as delineated by the Structure Commission of the International Zeolite Association refer to the most recent designation as of the priority date of this document unless otherwise indicated.

As used herein, the term weight percent (“wt %”) designates the percentage by weight a component is of a total weight of an indicated composition.

### Alcohol Ethoxylate

The present disclosure provides an alcohol ethoxylate having structure (I):

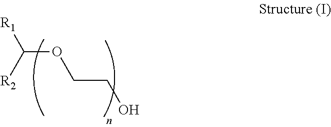

wherein R₁ is an alkyl, R₂ is selected from the group consisting of an alkyl group, and n has a value of 1 to 3. The alkyl of R₁ may have from one carbon atom (i.e., be a C₁ alkyl) to sixteen carbon atoms (i.e., be a C₁₆ alkyl). For example, R₁ may be a C₁ alkyl, or a C₂ alkyl, or a C₃ alkyl, or a C₄ alkyl, or a C₅ alkyl, or a C₆ alkyl, or a C₇ alkyl, or a C₈ alkyl, or a C₉ alkyl, or a C₁₀ alkyl, or a C₁ alkyl, or a C₁₂ alkyl, or a C₁₃ alkyl, or a C₁₄ alkyl, or a C₁₅ alkyl, or a C₁₁ alkyl. The alkyl of R₁ may be saturated or unsaturated. The alkyl of R₂ may be a C₁ to C₁₆ alkyl. For example, R₂ may be a C₁ alkyl, or a C₂ alkyl, or a C₃ alkyl, or a C₄ alkyl, or a C₅ alkyl, or a C₆ alkyl, or a C₇ alkyl, or a C₈ alkyl, or a C₉ alkyl, or a C₁₀ alkyl, or a C₁₁ alkyl, or a C₁₂ alkyl, or a C₁₃ alkyl, or a C₁₄ alkyl, or a C₁₅ alkyl, or a C₁₆ alkyl. The alkyl of R₂ may be saturated or unsaturated. The sum of carbon atoms present in R₁ and R₂ may be from 7 to 17. For example, the sum of carbon atoms present in R₁ and R₂ may be 7 or greater, or 8 or greater, or 9 or greater, or 10 or greater, or 11 or greater, or 12 or greater, or 13 or greater, or 14 or greater, or 15 or greater, or 16 or greater, while at the same time, 17 or less, or 16 or less, or 15 or less, or 14 or less, or 13 or less, or 12 or less, or 11 or less, or 10 or less, or 9 or less, or 8 or less. n of Structure (I) has a value from 1 to 3. For example, n may be 1, 2, or 3. The number of carbons present in R₁ and R₂ and the value of n are all determined according to ¹³C nuclear magnetic resonance characterization provided below.

Due to the natural distribution of products resulting from processes employed in forming the alcohol ethoxylates of Structure (I), oligomers of Structure (I) can vary in the value that n has. For example, a composition of oligomers of Structure (I) may have the same R₁ and R₂, but different n values of 1, 2 and 3. As a whole, 95 mol % or greater of the oligomers of Structure (I) have an n of 1 and 5 mol % or less of the oligomers of Structure (I) have an n of 2 or greater. For example, 95.0 mol % or greater, or 95.5 mol % or greater, or 96.0 mol % or greater, or 96.5 mol % or greater, or 97.0 mol % or greater, or 97.5 mol % or greater, or 98.0 mol % or greater, or 98.5 mol % or greater, or 99.0 mol % or greater, or 99.5 mol % or greater, while at the same time, 100.0 mol % or less, or 99.5 mol % or less, or 99.0 mol % or less, or 98.5 mol % or less, or 98.0 mol % or less, or 97.5 mol % or less, or 97.0 mol % or less, or 96.5 mol % or less, or 96.0 mol % or less, or 95.5 mol % or less of the oligomers of Structure (I) have an n value of 1 as determined according to ¹³C nuclear magnetic resonance characterization provided below. Further, 5.0 mol % or less, or 4.5 mol % or less, or 4.0 mol % or less, or 3.5 mol % or less, or 3.0 mol % or less, or 2.5 mol % or less, or 2.0 mol % or less, or 1.5 mol % or less, or 1.0 mol % or less, or 0.5 mol % or less, while at the same time, 0.0 mol % or greater, or 0.5 mol % or greater, or 1.0 mol % or greater, or 1.5 mol % or greater, or 2.0 mol % or greater, or 2.5 mol % or greater, or 3.0 mol % or greater, or 3.5 mol % or greater, or 4.0 mol % or greater, or 4.5 mol % or greater of the oligomers of Structure (I) have an n value of 2 or greater as determined according to ¹³C nuclear magnetic resonance characterization provided below.

### Surfactant Composition

The present disclosure is also directed to a surfactant composition. The surfactant composition comprises oligomers of a surfactant having structure (II):

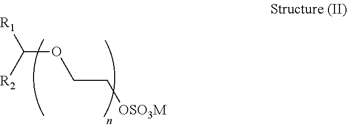

wherein R₁ is an alkyl and R₂ is an alkyl. The alkyl of R₁ may be a C₁ to C₁₆ alkyl. For example, R₁ may be a C₁ alkyl, or a C₂ alkyl, or a C₃ alkyl, or a C₄ alkyl, or a C₅ alkyl, or a C₆ alkyl, or a C₇ alkyl, or a C₈ alkyl, or a C₉ alkyl, or a C₁₀ alkyl, or a C₁₁ alkyl, or a C₁₂ alkyl, or a C₁₃ alkyl, or a C₁₄ alkyl, or a C₁₅ alkyl, or a C₁₆ alkyl. The alkyl of R₁ may be saturated or unsaturated. The alkyl of R₂ may be a C₁ to C₁₆ alkyl. For example, R₂ may be a C₁ alkyl, or a C₂ alkyl, or a C₃ alkyl, or a C₄ alkyl, or a C₅ alkyl, or a C₆ alkyl, or a C₇ alkyl, or a C₈ alkyl, or a C₉ alkyl, or a C₁₀ alkyl, or a C₁₁ alkyl, or a C₁₂ alkyl, or a C₁₃ alkyl, or a C₁₄ alkyl, or a C₁₅ alkyl, or a C₁₆ alkyl. The alkyl of R₂ may be saturated or unsaturated. The sum of carbon atoms present in R₁ and R₂ may be from 7 to 17. For example, the sum of carbon atoms present in R₁ and R₂ may be 7 or greater, or 8 or greater, or 9 or greater, or 10 or greater, or 11 or greater, or 12 or greater, or 13 or greater, or 14 or greater, or 15 or greater, or 16 or greater, while at the same time, 17 or less, or 16 or less, or 15 or less, or 14 or less, or 13 or less, or 12 or less, or 11 or less, or 10 or less, or 9 or less, or 8 or less. In a specific example, a sum of carbon atoms in R₁ and R₂ of Structure (II) is from 11 to 13. The n value of Structure (II) has a value from 1 to 3. For example, n may be 1, 2, or 3. The number of carbons present in R₁ and R₂ and the value of n are all determined according to ¹³C nuclear magnetic resonance (“NMR”) characterization provided below.

M of Structure (II) is selected from the group consisting of a proton, an ammonium cation, a metal cation, a nitrogen cation, a boron cation, a phosphorus cation, triethylamine, triethanolamine, monoethanolamine and combinations thereof. It will be understood that different surfactant molecules may have different materials for M.

As with Structure (I), Structure (II) can vary in the value that n has. In the surfactant composition, 95 mol % or greater of the oligomers of Structure (II) have an n of 1 and 5 mol % or less of the oligomers of Structure (II) have an n of 2 or greater. For example, 95.0 mol % or greater, or 95.5 mol % or greater, or 96.0 mol % or greater, or 96.5 mol % or greater, or 97.0 mol % or greater, or 97.5 mol % or greater, or 98.0 mol % or greater, or 98.5 mol % or greater, or 99.0 mol % or greater, or 99.5 mol % or greater, while at the same time, 100.0 mol % or less, or 99.5 mol % or less, or 99.0 mol % or less, or 98.5 mol % or less, or 98.0 mol % or less, or 97.5 mol % or less, or 97.0 mol % or less, or 96.5 mol % or less, or 96.0 mol % or less, or 95.5 mol % or less of the oligomers of Structure (II) in the composition have an n value of 1. Further, 5.0 mol % or less, or 4.5 mol % or less, or 4.0 mol % or less, or 3.5 mol % or less, or 3.0 mol % or less, or 2.5 mol % or less, or 2.0 mol % or less, or 1.5 mol % or less, or 1.0 mol % or less, or 0.5 mol % or less, while at the same time, 0.0 mol % or greater, or 0.5 mol % or greater, or 1.0 mol % or greater, or 1.5 mol % or greater, or 2.0 mol % or greater, or 2.5 mol % or greater, or 3.0 mol % or greater, or 3.5 mol % or greater, or 4.0 mol % or greater, or 4.5 mol % or greater of the oligomers of Structure (II) in the composition have an n value of 2 or greater.

The surfactant composition may have a 1,4 dioxane content of 9 ppm or less as measured according to the GCMS and LCMS method laid out in greater detail below. For example, the surfactant composition may exhibit a 1,4 dioxane of 9 ppm or less, or 8 ppm or less, or 7 ppm or less, or 6 ppm or less, or 5 ppm or less, or 4 ppm or less, or 3 ppm or less, or 2 ppm or less, or 1 ppm or less. As explained above, the relative molar concentrations of the oligomers of Structures (I) and (II) mean that the formation of dioxane is not only resisted during the sulfation process, but also in the presence of heat and after the passing of time.

### Material

The present disclosure is also directed to a material comprising the surfactant composition. The material may be a personal care product, a cleaning product, a coating, an emulsion polymerization solution, a textile process additive or composition, an agricultural adjuvant or composition, an oil and gas production additive or composition, an ink, a paper and pulp additive or composition, and other industrial processes additive or composition. The material may comprise from 0.1 weight percent (“wt %”) to 99.9 wt % of the surfactant composition based on a total weight of the material. For example, the material may comprise 0.1 wt % or greater, or 1.0 wt % or greater, or 5.0 wt % or greater, or 10 wt % or greater, or 20 wt % or greater, or 30 wt % or greater, or 40 wt % or greater, or 50 wt % or greater, or 60 wt % or greater, or 70 wt % or greater, or 80 wt % or greater, or 90 wt % or greater, or 99 wt % or greater, while at the same time, 99.9 wt % or less, or 99 wt % or less, or 90 wt % or less, or 80 wt % or less, or 70 wt % or less, or 60 wt % or less, or 50 wt % or less, or 40 wt % or less, or 30 wt % or less, or 20 wt % or less, or 10 wt % or less, or 5 wt % or less, or 1.0 wt % or less of the surfactant composition based on a total weight of the material. Similarly to the surfactant composition, the material may have a 1,4 dioxane content of 9 ppm or less as measured according to the headspace gas chromatography with flame ionization detection procedure laid out in greater detail below.

### Process

The present disclosure is also directed to a process. The process includes a step of contacting an olefin, an alcohol and a metallosilicate catalyst to form oligomers of an alcohol ethoxylate having Structure (I).

Contacting the olefin, alcohol, metallosilicate catalyst and solvent result in the generation of an alcohol ethoxylate having Structure (I). The chemical reaction between the olefin and the alcohol is catalyzed by the metallosilicate catalyst in a reactor to generate the alcohol ethoxylate having Structure (I).

The reaction of the olefin and the alcohol may take place at a temperature from 50° C. to 300° C. or from 100° C. to 200° C. In a specific example, the reaction may be carried out at 135° C.

Reaction of the olefin and the alcohol may be carried out in a batch reactor, continuous reactor or fixed-bed reactor. In operation of the chemical reaction, the Brøsnsted acid sites of the metallosilicate catalyst catalyze the etherification of the olefin to the alcohol through an addition type reaction. The reaction of the olefin and the alcohol produces the alcohol ethoxylate having Structure (I).

### Olefin

The olefin used in the process may be linear, branched, acyclic, cyclic, or mixtures thereof.

The olefin may be a C₈ to C₁₈ olefin. The olefin may be a C₈ olefin, or a C₉ olefin, or a C₁₀ olefin, or a C₁₁ olefin, or a C₁₂ olefin, or a C₁₃ olefin, or a C₁₄ olefin, or a C₁₅ olefin, or a C₁₆ olefin, or a C₁₇ olefin, or a C₁₈ olefin.

The olefin may include alkenes such as alpha (α) olefins, internal disubstituted olefins, or cyclic structures (e.g., C₈-C₁₂ cycloalkene). α olefins include an unsaturated bond in the α-position of the olefin. Suitable α olefins may be selected from the group consisting of 1-octene, 1-decene, 1-dodecene, 1-tetradecene, 1-hexadecene, 1-octadecene, 1-icosene, 1-docosene and combinations thereof. Internal disubstituted olefins include an unsaturated bond not in a terminal location on the olefin. Internal olefins may be selected from the group consisting of 2-octene, 3-octene, 4-octene, 2-nonene, 3-nonene, 4-nonene, 2-decene, 3-decene, 4-decene, 5-decene and combinations thereof. Other exemplary olefins may include butadiene and styrene.

Examples of suitable commercially available olefins include NEODENE™ 8, NEODENE™ 10, NEODENE™ 12, NEODENE™ 14, NEODENE™ 16, NEODENE™ 1214, NEODENE™ 1416, NEODENE™ 16148 from Shell, The Hague, Netherlands.

### Alcohol

The alcohol utilized in the process is an alkylene glycol. The alcohol may be selected from the group consisting of monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof. According to various examples, the alcohol is a (poly)alkylene glycol such as monoethylene glycol, diethylene glycol, propylene glycol and triethylene glycol.

A molar ratio of alcohol to olefin in the process may be from be 20:1 or less, or 15:1 or less, or 10:1 or less, or 9:1 or less, or 8:1 or less, or 7:1 or less, or 6:1 or less, or 5:1 or less, or 4:1 or less, or 3:1 or less, or 2:1 or less, or 0.2:1 or less, while at the same time, 0.1:1 or greater, or 1:1 or greater, or 1:2 or greater, or 1:3 or greater, or 1:4 or greater, or 1:5 or greater, or 1:6 or greater, or 1:7 or greater, or 1:8 or greater, or 1:9 or greater, or 1:10 or greater, or 1:15 or greater, or 1:20 or greater.

### Metallosilicate Catalyst

As used herein the term “metallosilicate catalyst” is an aluminosilicate (commonly referred to as a zeolite) compound having a crystal lattice that has had one or more metal elements substituted in the crystal lattice for a silicon atom. The crystal lattice of the metallosilicate catalyst form cavities and channels inside where cations, water and/or small molecules may reside. The substitute metal element may include one or more metals selected from the group consisting of B, Al, Ga, In, Ge, Sn, P, As, Sb, Sc, Y, La, Ti, Zr, V, Cr, Mn, Pb, Pd, Pt, Au, Fe, Co, Ni, Cu, Zn. The metallosilicate catalyst may be substantially free of Hf. According to various examples, the metallosilicate may have a silica to alumina ratio of from 5:1 to 1,500:1 as measured using Neutron Activation Analysis. The silica to alumina ratio may be from 5:1 to 1,500:1, or from 10:1 to 500:1, or from 10:1 to 400:1, or from 10:1 to 300:1 or from 10:1 to 200:1. Such a silica to alumina ratio may be advantageous in providing a highly homogenous metallosilicate catalyst with an organophilic-hydrophobic selectivity that adsorb non-polar organic molecules.

The metallosilicate catalyst may have one or more ion-exchangeable cations outside the crystal lattice. The ion-exchangeable cation may include H⁺, Li⁺, Na⁺, Rb⁺, Cs⁺, Mg²⁺, Ca²⁺, Sr²⁺, Ba²⁺, Sc³⁺, Y³⁺La³⁺, R₄N⁺, R₄P⁺ (where R is H or alkyl).

The metallosilicate catalyst may take a variety of crystal structures. Specific examples of the metallosilicate catalyst structures include MFI (e.g. ZSM-5), MEL (e.g. ZSM-11), BEA (e.g. (3-type zeolite), FAU (e.g. Y-type zeolite), MOR (e.g. Mordenite), MTW (e.g. ZSM-12), and LTL (e.g. Linde L), as described using IUPAC codes in accordance with nomenclature by the Structure Commission of the International Zeolite Association.

The crystalline frameworks of metallosilicate catalyst are represented by networks of molecular-sized channels and cages comprised of corner-shared tetrahedral [TO₄] (T=Si or Al) primary building blocks. A negative charge can be introduced onto the framework via the isomorphous substitution of a framework tetravalent silicon by a trivalent metal (e.g., aluminum) atom. The overall charge neutrality is then achieved by the introduction of cationic species compensating for the resulting negative lattice charge. When such a charge-compensation is provided by protons, Brönsted acid sites are formed rendering the resulting H-forms of zeolites strong solid Brønsted acids.

The metallosilicate catalysts may be used in the method in a variety of forms. For example, the metallosilicate catalysts may be powdered (e.g., particles having a longest linear dimension of less than 100 micrometers), granular (e.g., particles having a longest linear dimension of 100 micrometers or greater), or molded articles of powdered and/or granular metallosilicate catalysts.

The metallosilicate catalysts may have a surface area of 100 m²/g or greater, or 200 m²/g or greater, or 300 m²/g or greater, or 400 m²/g or greater, or 500 m²/g or greater, or 600 m²/g or greater, or 700 m²/g or greater, or 800 m²/g or greater, or 900 m²/g or greater, while at the same time, 1000 m²/g or less, or 900 m²/g or less, or 800 m²/g or less, or 700 m²/g or less, or 600 m²/g or less, or 500 m²/g or less, or 400 m²/g or less, or 300 m²/g or less, or 200 m²/g or less. Surface area is measured according to ASTM D4365-19.

Metallosilicate catalysts can be synthesized by hydrothermal synthesis methods. For example, the metallosilicate catalysts can be synthesized from heating a composition comprising a silica source (e.g., silica sol, silica gel, and alkoxysilanes), a metal source (e.g., metal sulfates, metal oxides, metal halides, etc.), and a quaternary ammonium salt such as a tetraethylammonium salt or tetrapropylammonium to a temperature of about 100° C. to about 175° C. until a crystal solid forms. The resulting crystal solid is then filtered off, washed with water, and dried, and then calcined at a temperature form 350° C. to 600° C.

Examples of suitable commercially available metallosilicate catalysts include CP814E, CP814C, CP811C-300, CBV 712, CBV 720, CBV 760, CBV 2314, CBV 10A from ZEOLYST INTERNATIONAL™ of Conshohocken, PA.

### Sulfation

After formation of the oligomers of Structure (I), a step of sulfating the oligomers of Structure (I) to form oligomers of Structure (II) is performed. Sulfation of the alcohol ethoxylate having Structure (I) is accomplished by contacting the alcohol ethoxylate with a sulfating agent that can sulfate an alcohol using a known process as described in references (David W. Roberts, “Sulfonation Technologies for Anionic Surfactant Manufacture”, Organic Process Research & Development 1998, 2, 194-202; Xavier Domingo, “Alcohol and Alcohol Ether Sulfates”, in Anionic Surfactants (Organic Chemistry), Surfactant Science Series Vol. 56 (Helmut W. Stache, ed.), Marcel Dekker, Inc., New York, 1996). Useful sulfating agents include chlorosulfonic acid, sulfuric acid, sulfur trioxide, sulfamic acid. When chlorosulfonic acid is used as the sulfating agent, the sulfation is performed at a temperature from −10° C. to 10° C. and at a pressure of 0.01 megapascals (“MPa”) to 1 MPa. The sulfating agent may be metered in or combined with the alcohol ethoxylate all at once. Once the desired level of sulfation of the alcohol ethoxylate is reached, a base (e.g., NaOH, KOH, or NH₄OH) may be used to neutralize the sulfate or sulfate bearing compound and end the sulfation reaction.

### Examples

### Materials

Catalyst is a metallosilicate catalysts defined by a BEA structure and having a silica to alumina ratio of 25:1 and a surface area of 680 m²/g, that is commercially available as CP814E from ZEOLYST INTERNATIONAL™ of Conshohocken, PA.

1-Dodecene is an alpha olefin that is commercially available as NEODENE™ 12 from the SHELL™ group of The Hague, Netherlands.

1-Tetradecene is an alpha olefin that is commercially available as NEODENE™ 14 from the SHELL™ group of The Hague, Netherlands.

Monoethylene Glycol is liquid anhydrous ethylene glycol purchased from SIGMA ALDRICH™ having a CAS Number of 107-21-1.

ALEO1 is linear primary alcohol ethoxylate with an average of 1 ethylene oxide unit as measured according to ¹³C NMR characterization that is commercially available under the tradename SURFONIC™ L24-1 from Huntsman Corporation, The Woodlands, Texas.

ALEO2 is a 7 mole ethoxylate of linear primary C₁₂-C₁₅ alcohols that is commercially available under the tradename BIOSOFT™ N25-7 from Stepan Company, Northfield, Illinois.

Sulfuric Acid is a ACS reagent (95-98%) from SIGMA ALDRICH™ having a CAS number of 7664-93-9.

Chlorosulfonic Acid is a liquid purchased from SIGMA ALDRICH™ having a CAS number of 7790-94-5.

Sodium Hydroxide (NaOH) is a solid purchased from SIGMA ALDRICH™ having a CAS number of 1310-73-2.

Dichloromethane (DCM) is a liquid anhydrous dichloromethane purchased from SIGMA ALDRICH™ having a CAS number 75-09-2.

SA3EO sulfate is a secondary alcohol ethoxylate with 3 ethylene oxide units that has been sulfated in the same manner as described below in connection with the C₁₂EO sulfate. The base starting material is a C₁₂-C₁₄-secondary alcohol ethoxylate having a CAS number of 84133-50-6 and which is commercially available as TERGITOL™ 15-S-3 from The Dow Chemical Company, Midland, Michigan.

Sodium Dioctyl Sulfosuccinate (SDOSS) BioXtra ≥99.0% is a solid, purchased from SIGMA ALDRICH™ having a CAS number 577-11-7.

Sodium dodecyl Sulfate (SDS) ≥99.0% (GC) dust-free pellets, purchased from SIGMA ALDRICH™ having a CAS number 151-21-3.

Hyamine 1622 (Benzethonium Chloride) ≥99.0% (AT) is a solid, purchased from SIGMA ALDRICH™ having a CAS number 121-54-0.

Methylene Blue (Certified Biological Stain) is a solid, purchased from Fischer Scientific™ having a CAS number 7220-79-3.

Sodium Sulfate Anhydrous (Granular/Certified ACS), is a solid, purchased from Fischer Scientific™ having a CAS number 7757-82-6.

### Test Methods

### Method for Determining Percent Active Sulfate Content

This procedure is a modification of Turney, M. E. Cannell, D. W, Alkaline methylene blue method for determination of anionic surfactants and for amine oxides in detergents. J. Am. Oil Chem. Soc., 42, 544-546 (1965) and Epton. S. R. A new method for the rapid titrimetric analysis of sodium alkyl sulphates and related compounds. Trans. Faraday Soc., 44, 226-230 (1948).
1. Sodium Dioctyl sulfosuccinate (SDOSS), Control Solution—A 20 mL scintillation vial was loaded with 0.8 g (±0.1 mg) of sodium dioctyl sulfosuccinate of known purity (≥99%). The vial was filled with 10 mL of DI water and was transferred to a 250 mL volumetric flask. This process was repeated 3-5 times to ensure the sample was transferred. Once the transfer was complete, the sample was diluted with DI water to mark (250 mL). The flask was then stoppered, and the solution was thoroughly mixed by repeatedly inverting the flask.
   a. To verify accuracy, a second control solution can be made with sodium dodecyl sulfate (SDS), purity ≥99%, using a similar procedure as above. If a second control solution is used be sure to adjust equation 1 accordingly.
   b. Control sample(s) purity should be ≥99%. Equations listed below do not factor in purity, therefore if the control sample is below this it must be taken into account.
2. Methylene Blue chloride Solution—A 20 mL scintillation vial was loaded with 0.050 g (±0.005) of methylene blue chloride. The vial was filled with 10 mL of DI water and was transferred to a 1 L graduated cylinder. This process was repeated 3-5 times to ensure the sample was transferred. The methylene blue solution was then diluted to the 1 L mark. This solution was then transferred to a 2 L glass jug. Once transfer is complete, 10 mL of concentrated sulfuric acid and 50 g of anhydrous sodium sulfate is carefully added to the methylene blue solution and mixed thoroughly.
3. Hyamine 1622 Solution—A 20 mL scintillation vial was loaded with 1.1 grams of Hyamine 1622. The vial was filled with 10 mL of DI water and was transferred to a 1 L volumetric flask. This process was repeated 3-5 times to ensure the sample was transferred. The sample was then diluted to the 1 L mark with DI water. The solution was thoroughly mixed by repeatedly inverting the flask and was then filtered into a 1 L glass bottle.
4. Standardization of Hyamine solution—A 100 mL glass bottle was loaded with 5 mL of the sodium dioctyl sulfosuccinate solution via pipette. Next, 20 mL of methylene blue chloride solution and 25 mL of chloroform was added via pipette. The 100 mL bottle is capped and shaken thoroughly for several seconds. Hyamine 1622 solution was then titrated into the methylene blue/chloroform biphasic mixture at 0.5 mL increments. After each addition the bottle was capped and shaken vigorously. Continue the addition of Hyamine 1622, in 0.5 mL increments, until the blue color in the chloroform layer (or lower layer) begins to migrate to the upper layer. At this point, decrease addition of Hyamine 1622 to 0.1 mL and continue until the endpoint is reached. The endpoint is reached when the blue color intensity of both layers is matched. This standardization is completed in triplicate. The moles of the Hyamine 1622 was calculated by this formula:

$${Mol}_{H} = \frac{C {S}_{vol} ( mL ) * C {S}_{mass} ( g )}{{H}_{CSVol} ( mL ) * C {S}_{MW} ( \frac{g}{mol} )} \qquad ( Eq . 1 )$$

Mol_{H}=Moles of hyamine
CS_{Vol}=Volume of control solution being tested (5 mL)
CSₘₐₛₛ=Total grams of control (SDOSS) added to 250 mL control solution, to the nearest 0.1 mg (g)
H_{CSVol}=Amount of hyamine solution needed to reach the endpoint for control solution (mL)
CS_{MW}=molecular weight of control, SDOSS=444.56 (g/mol)
5. Procedure to Determine percent active sulfate content—A 20 mL scintillation vial was loaded with 0.9 to 1.1 grams of the sample, weighed to the nearest 0.1 mg. The vial was filled with 10 mL of DI water and was transferred to a 250 mL volumetric flask. This process was repeated 3-5 times to ensure the sample was transferred. Once the transfer was complete, the sample was diluted with DI water to mark (250 mL). The flask was then stoppered, and the solution was thoroughly mixed by repeatedly inverting the flask. Next, a 100 mL glass bottle was loaded with 5 mL of the test sample solution via pipette. The 100 mL glass bottle was then loaded with 20 mL of the methylene blue solution and 25 mL of chloroform via pipette. The glass bottle was capped and shaken vigorously for several seconds. Hyamine 1622 solution was then titrated into the methylene blue/chloroform biphasic mixture at 0.5 mL increments. After each addition the bottle was capped and shaken vigorously. Continue the addition of Hyamine 1622, in 0.5 mL increments, until the blue color in the chloroform layer (or lower layer) begins to migrate to the upper layer. At this point, decrease addition of Hyamine 1622 to 0.1 mL and continue until the endpoint is reached. The endpoint is reached when the color intensity of both layers is matched. Each sample is repeated in triplicate. The percent active sulfate content (wt %) was determined using by this formula:

$$\% Active Sulfate Content = \frac{{H}_{SVol} ( mL ) * {Mol}_{H} ( mol ) * {S}_{MW} ( \frac{g}{mol} )}{{S}_{Vol} ( mL ) * {S}_{Mass} ( g )} * 100 \qquad ( Eq . 2 )$$

H_{SVol}=Amount of Hyamine Solution needed to reach the endpoint for test sample solution (mL)
Mol_{H}=Moles of Hyamine (solved for in Eq. 1.)
S_{MW}=Average MW of test sample (g/mol)
S_{Vor}=Volume of sample solution being tested (5 mL)
Sₘₐₛₛ=Total grams of test sample added to 250 mL solution, to the nearest 0.1 mg (g)

### Procedure for Acid Digestion

Load a sample vial with 0.5 grams of the alcohol ethoxylate material to be tested. Next, concentrated sulfuric acid (4.0 grams, ACS reagent 95-98%) is added to form a reaction solution and the sample vial is tightly capped. The sample vials are then placed onto a heated block and the samples heated to 90° C. Once the set temperature is reached, the reaction is allowed to react for 1 hour. Once completed, the reaction sample vials are removed and allowed to cool to 23° C. While this sample is cooling, a separate gas chromatography head space vial is loaded with 0.9 grams of 1 molar aqueous NaOH solution and is cooled in an ice bath. After the reaction solution is cooled, 0.1 grams is added to the pre-cooled gas chromatography head space vial. The vial is capped immediately and placed back into the ice bath. This sample is then analyzed by headspace gas chromatography with flame ionization detection (“HS-GC/FID”) to determine the amount of 1,4-dioxane formed during the digestion. 1,4-dioxane will complex with sulfuric acid, thus the neutralization step is necessary to liberate any 1,4-dioxane generated during the sulfuric acid digestion of the samples.

### Acid Digestion Analysis

The neutralized headspace vials are heated at 90° C. for 15 minutes in order to allow the concentration of 1,4-dioxane to equilibrate into the headspace. A 2.5 ml aliquot of the headspace is extracted using a gas-tight syringe heated at 150° C. and subsequently injected into the gas chromatographic instrument. The volatile components in the headspace sample are separated using a Porabond Q column and then detected by a flame ionization detector. The Porabond Q column is used because it does not readily degraded under acidic or basic conditions and is able to provide the best separation and limit of detection of 1,4-dioxane amongst the other acid degraded sample matrix components. Quantitation is performed by external standardization and the method is found to have a limit of detection of 0.1 ppm (w/w) for 1,4-dioxane.

| GC conditions | | |
|---|---|---|
| GC system | Agilent 7890 GC system | |
| Analytical column | DB-1 30 m × 0.32 mm × 5 um | |
| Inlet Type | Split/Splitless | |
| Inlet Pressure | 10.95 | psi |
| Total flow | 17.2 | mL/min |
| Septum purge flow | 4 | mL/min |
| Split Ratio | 10:1 | |
| Inlet Temperature | 110° | C. |
| Injection Volume | 1 | μL |
| Column flow | 1.2 mL/min, constant flow | |
| GC Oven | 35° C. hold for 2.00 min, 10° C./min to 300° | |
| | C. hold for 5.00 min | |
| GC Run Time | 33.50 | minutes |
| MS System | Agilent 5977A MSD | |
| MS Parameters | | |
| Mode | EI | |
| Transfer line temperature | 250° | C. |
| MS Source | 230° | C. |
| MS Quad | 150° | C. |
| Acquisition Mode | SIM | |
| SIM Ion | 88.00 | m/z |
| Dwell Time | 250 | |
| Mass range | 33-750 | m/z |
| Energy | 70 | eV |
| Emission Current | 34.6 | mA |

| | |
|---|---|
| Solvent delay | None |

Standards were prepared by adding dioxane in tetrahydrofuran (“THF”) and diluting down to 0.1-100 ppm.

Samples were prepared by mixing 3.3 g from the organic (DCM) layer of the crude process mixture and 6.7 g of THF, the solution was then allowed to shake for about 20 minutes. The solids were then centrifuged to the bottom and the supernatant was vialed in an autosampler vial. Spiked samples were prepared by spiking dioxane standard in THF into separate samples at 5-10 ppm.

### LCMS Method for Aqueous Layer

| Instrument | Agilent 1260 Infinity Series w/6470 Triple Quad MS | | |
|---|---|---|---|
| Column | Waters Atlantis T3 3.0 × 150 mm, 3 μm | | |
| Column temp. | 30° C. | | |
| Mobile Phase | A: Water | | |
| Solvents: | B: Acetonitrile | | |
| Flow rate | 0.40 mL/min | | |
| | Time | | |
| LC Conditions | (mins) | Pump A | Pump B |
| | 0 | 95% | 5% |
| | 1 | 95% | 5% |
| | 16 | 60% | 40% |
| | 17 | 5% | 95% |
| | 22 | 5% | 95% |

| | |
|---|---|
| Run Time | 22.00 mins |
| Post Time | 8 mins |
| Injection Volume | 5 μL |
| UV wavelength | 190-600 nm |
| APCI conditions | Spray Voltage: 3500 V (+) |
| | Gas Temp.: 350° C. |
| | APCI Heater Temp: 500° C. |
| | Gas Flow: 9 L/min |
| | Nebulizer: 60 psi |
| | Fragmentor: 77 |
| MS Parameters | Mode: SIM |
| | SIM Ion: 89.2 m/z |
| | Dwell Time: 500 msec |

Samples were injected neat or diluted with water 1:4. Standards were prepared by preparing a dioxane in THF stock solution and the diluting down with water to 0.1-100 ppm.

### Calculation of Dioxane Content Relative to Solids

The ppm of dioxane content relative to solids content in the sample is calculated according to equation 3.

$$\frac{Concentration of Dioxane in Solution ( \frac{mg}{L} or p p M ) * Total Reaction Volume ( L )}{Theoretical Yield of product ( mg )} \times {10}^{6} \qquad ( Eq . 3 )$$

### Calculation for EO Content

The weight percent of ethylene oxide content in the sample is calculated according to equation 2 where MW is the molecular weight in g/mol.

$$\frac{0.1 * ( Moles of EO ) * ( MW of EO )}{Amount of alcohol ethoxylate material in grams + amount of sulfuric acid in grams} \qquad ( Eq . 4 )$$

### Nuclear Magnetic Resonance (“NMR”) Characterization for EO Distribution

¹³C NMR spectra of the samples dissolved in deuterated dimethyl sulfoxide containing 0.025M chromium (III) acetylacetonate are collected on a Bruker AVANCE 400 MHz spectrometer equipped with a 10 mm cryo-probe set to 25° C. The spectra are acquired with the following parameters: a 90°-pulse, inverse-gated decoupling, a 1.38 second acquisition time, and a 6.4 second recycle delay. 2048 scans were collected. The data is processed in MNOVA, and the chemical shifts are referenced to the solvent peak at 39.52 ppm. A DEPT-135 experiment is also acquired with the same parameters, but with a 2.0 second recycle delay, and 2048 scans.

The ratios of different EO adducts are calculated by integrating and comparing the intensity of the ethylene oxide alcohol end groups from about 60-61 ppm, the ethylene oxide backbone groups from about 69-70 ppm, the ethylene oxide end group ether peak from about 71-72 ppm, the unreacted primary alcohol peaks from about 60-61 ppm, and the unreacted secondary alcohol peaks from about 65-66 ppm.

### Sample Preparation

### Synthesis of C₁₂EO

A 3 liter (“L”) 3-neck glass round bottom flask, equipped with an overhead stirred through the center neck, reflux condenser and-a heating jacket was used for the etherification of 1-dodecene and monoethylene glycol with the catalyst. To ensure good mixing, a pitch blade impeller was used for agitation. A reaction mixture of 551.7-grams (“g”) ethylene glycol and 505.8 g 1-dodecene was prepared and loaded in the reactor together with 61-g catalyst in powdered form at 23° C. The impeller stirring rate was set to be at 400 revolutions per minute (“rpm”). The reactor was heated to 135° C. in over the course of 30 minutes, held at 135° C. for 18 hours and then the reactor was cooled down to 23° C. by shutting off the heater. The reaction mixture was separated into a monoethylene glycol and catalyst phase and an olefin phase using a separation funnel.

A distillation apparatus was constructed using a 1-liter round bottom flask connected to a short path distillation head with a thermometer adapter and a condenser with a vacuum adapter at the outlet. The distillation flask was heated in an aluminum block by an IKA heated stir-plate. The distillation pot was charged with the combined olefin phase and then stirring and vacuum were applied. Significant boiling was observed but no condensate was observed or collected. The temperature of the heating block was raised to 75° C. and unreacted dodecane was collected at a distillation head temperature of 25° C. to 50° C. and a pressure of 13.3-40 pascals (“Pa”). The heating block temperature was raised gradually to 140° C. and an intermediate fraction containing both monoether alcohol ethoxylates and dodecenes was recovered while the head temperature increased from 50° C. to 75° C. at a pressure of 13 Pa. The C₁₂EO was collected at ahead temperature of 70° C. to 115° C. and a pressure of 6 Pa to 33 Pa. The heating block temperature was raised gradually to 200° C. and an intermediate fraction containing both monoether alcohol ethoxylates and diether was collected while the head temperature increased from 115° C. to 130° C. at a pressure of 6 Pa. The distillation was discontinued and the diether, which remained in the pot, was collected. The C₁₂EO sample was characterized by NMR to understand the product compositions and is reported in the results section. C₁₂EO was carried to the next sulfation process to make sulfate anionic surfactants.

### Preparation of C₁₂EO Sulfate and ALEO1 Sulfate

All chemical manipulations were conducted under a dry nitrogen atmosphere. Prior to the experiment all glassware was heated in a laboratory oven to remove residual water. A 2-L three-neck round bottom flask was loaded with dichloromethane (500 mL) and C₁₂EO (40 g, 0.173 mol, 1.0 equivalents). The reaction flask was equipped with an overhead mechanical stirrer, additional funnel, and thermocouple. Next, chlorosulfonic acid (12.7 mL, 0.191 mol, 1.1 equivalents) was carefully loaded into the additional funnel. The reaction flask was then submerged into an ice-bath and allowed to cool for 20 minutes, down to 0° C. Once the reaction was cooled, chlorosulfonic acid was added to the reaction flask dropwise, at a rate of approx. 1.0 mL per minute, over approx. 20 minutes. During the addition of chlorosulfonic acid the reaction temperature did not exceed 5° C. After the addition, the reaction was allowed to react, and the temperature was kept between 0 and 5° C., for 3 hours. At this time, the reaction was neutralized by slow dropwise addition of an aqueous NaOH solution (18.0 g NaOH in 500 mL of water, 0.9 molar). The rate of addition was slow enough to not exceed 5° C. over the course of addition. The solution became basic after the addition of ˜300 mL of 0.9 molar NaOH solution. Dichloromethane was then carefully removed from the biphasic reaction in vacuo. During the removal of the dichloromethane, a large amount of foam was observed. Upon removal of the dichloromethane, the remaining aqueous solution was placed in a freeze drier/lyophilizer to yield the secondary alcohol ethoxylate sulfate product, C₁₂EO Sulfate, as a whiteish solid (61.9 grams). The secondary alcohol ethoxylate sulfate product was 79.9 wt % actives as measured according to the Method for determining Percent Active Sulfate Content.

The synthesis of the ALEO1 sulfate and SA3EO sulfate was performed in the same manner as the C₁₂EO sulfate.

### Synthesis of C₁₄EO

A 300 mL Parr reactor with a heating jacket and controller was used for the etherification of 1-tetradecene and monoethylene glycol with a catalyst. To ensure good mixing, a pitch blade impeller was used for agitation.

The reaction mixture of 100.0 g monoethylene glycol and 100.0 g 1-tetradecene was prepared and loaded in the reactor together with 10.0 g powder form catalyst at 23° C. The impeller stirring rate was set to be at least 600 rpm. The reactor was heated up to 135° C. in 30 minutes, held at 135° C. for 6 hours and then the reactor was cooled down to room temperature by shutting off the heater. The reaction mixture was separated by a separation funnel. The reaction mixture was separated into a monoethylene glycol and catalyst phase and an olefin phase using a separation funnel. Fifteen batches were generated and the olefin phases were collected and combined for distillation.

The same distillation apparatus as used in the synthesis of C₁₂EO was used for distillation of the C₁₄EO. The distillation pot was charged with the products in the olefin phase from multiple batch reactor runs and then stirring and vacuum were applied. Significant boiling was observed but no condensate was observed or collected. The temperature of the heating block was raised to 95° C. and unreacted 1-tetradecene was collected at a distillation head temperature of 30° C. to 60° C. at a pressure of 27 Pa to 5 Pa. The heating block temperature was raised gradually to 170° C. and an intermediate fraction containing both monoether and tetradecene was recovered while the head temperature increased from 60° C. to 85° C. at a pressure of 7 Pa to 5 Pa. The C₁₄EO was collected at a head temperature of 80° C. to 115° C. and a pressure of 8 Pa to 5 Pa. The distillation was discontinued when no more material would distill over with the pot temperature set at 170° C. The C₁₄EO sample was characterized by NMR to understand the product compositions.

### Preparation of C₁₄EO Sulfate

All chemical manipulations were conducted under a dry nitrogen atmosphere. Prior to the experiment all glassware was heated in a laboratory oven to remove residual water. A 2-L three-neck round bottom flask was loaded with dichloromethane (500 mL) and C₁₄EO (50 g, 0.193 mol, 1.0 equivalents). The reaction flask was equipped with an overhead mechanical stirrer, additional funnel, and thermocouple. Next, chlorosulfonic acid (14.2 mL, 0.213 mol, 1.1 equivalents) was carefully loaded into the additional funnel. The reaction flask was then submerged into an ice-bath and allowed to cool for 20 minutes, down to 0° C. Once the reaction was cooled, chlorosulfonic acid was added to the reaction flask dropwise, at a rate of approximately 1.0 mL per minute, over approximately 20 minutes. During the addition of chlorosulfonic acid the reaction temperature did not exceed 5° C. After the addition, the reaction was allowed to react and the temperature was kept between 0° C. and 5° C., for 3 hours. At this time, the reaction was neutralized by slow dropwise addition of aqueous NaOH (18.0 g in 500 mL of water, 0.9 molar). The rate of addition was slow enough to not exceed 5° C. over the course of addition. The solution became basic after the addition of about 400 mL of 0.9 molar NaOH solution. Dichloromethane was then carefully removed from the biphasic reaction in vacuo. During the removal of DCM, a large amount of foam was observed. Upon removal of DCM, the remaining aqueous solution was placed in a freeze drier/lyophilizer to give the secondary alcohol ethoxylate sulfate product (68.6 grams). The secondary alcohol ethoxylate sulfate product was 87.9 wt % actives.

### General Procedure for Conducting and Measuring the Dioxane Content Formed During Sulfation

Sulfations were conducted using similar conditions for the preparation of the C12EO and C14EO sulfate materials. A 2-L three-neck round bottom flask was loaded with 400-500 mL of dichloromethane and 35 to 50 grams (1 equiv.) of the alcohol ethoxylate being tested. The reaction flask was equipped with an overhead mechanical stirrer, additional funnel, and thermocouple. Next, chlorosulfonic acid (1.1 equiv.) was carefully loaded into the additional funnel. The reaction flask was then submerged into an ice-bath and allowed to cool for 20 minutes, down to 0° C. Once the reaction was cooled, chlorosulfonic acid was added to the reaction flask dropwise, at a rate of approx. 1.0 mL per minute, over approx. 20 minutes. During the addition of chlorosulfonic acid the reaction temperature did not exceed 5° C. After the addition, the reaction was allowed to react and the temperature was kept between 0 and 5° C., for 3 hours. At this time, the reaction was neutralized by slow dropwise addition of NaOH aq. (18.0 g in 500 mL of water, 0.9 M). The rate of addition was slow enough to not exceed 5° C. over the course of addition. Once neutralized, the biphasic mixture was analyzed by the GC method to determine the dioxane content formed during sulfation. The crude samples were tightly sealed and kept in a refrigerator until the analytical analysis was conducted.

### Results

### Alcohol Ethoxylates

The results of the NMR testing are provided in Table 1.

TABLE 1

| | EO adduct (molar %) | | |
|---|---|---|---|
| Material | 0 | 1 | >=2 |
| ALEO1 | 51.2 | 2.0 | 46.8 |
| C₁₂EO | 1.0 | 98.0 | 1.0 |
| C₁₄EO | 1.0 | 98.0 | 1.0 |

As expected, 95 mol % or greater of the oligomers of Structure (I) in the C₁₂EO and C₁₄EO have an n of 1 and 5 mol % or less of the oligomers of Structure (I) in the C₁₂EO and C₁₄EO have an n of 2 or greater. Specifically, 98 mol % or greater of the oligomers of Structure (I) in the C₁₂EO and C₁₄EO have an n of 1 and 2 mol % or less of the oligomers of Structure (I) in the C₁₂EO and C₁₄EO have an n of 2 or greater.

Table 2 provides the results of the acid digestion test in comparative examples (“CE”) and an inventive example (“IE”).

TABLE 2

| | | | | Conversion |
|---|---|---|---|---|
| | | EO content | 1,4-dioxane | of EO to |
| | Sample | (wt %) | generated (wt %) | DO, % |
| CE1 | ALEO2 | 6.9 | 2.53 | 37 |
| CE2 | ALEO1 | 2.2 | 0.35 | 16 |
| IE1 | C₁₂EO | 2.1 | 0.00052 | 0.02 |

Given the understanding above about how dioxane is believed to be formed, it is important to know the stability of alcohol ethoxylates under acidic environments such as the H₂SO₄ acidic digestion experiment. CE1 demonstrates that alcohol ethoxylates having EO content of 2 or greater easily generate dioxane under conditions like the acid digestion test. Under the acidic conditions, 2.53 wt % (25,300 ppm) dioxane was observed and 37% of the EO was converted to 1,4-dioxane.

CE2 has an average of 1EO, however, CE2 contained a significant amount of unreacted alcohol and 2EO adducts (i.e., 0EO and ≥2EO, but less 1EO). As explained above, alcohol ethoxylates having ≥2EO provide a chemical path for the formation of 1,4-dioxane in acidic conditions. Accordingly, it is unsurprising that CE2 shows 0.35 wt % (3500 ppm) dioxane and 16% EO conversion to 1,4-dioxane. ALEO1 and ALEO2 of CE1 and CE2 would clearly therefore face issues with respect to dioxane regulation if used in products and exposed to sulfur bearing acidic compounds.

C₁₂EO as IE1 was also tested in the acid digestion study. After acid digestion, the 1,4-dioxane content was 0.00052 wt % (5.2 ppm) which confirms that compositions that are rich (i.e., ≥95 mol %) in 1EO alcohol ethoxylates exhibit better stability under acidic conditions thereby preventing 1,4-dioxane formation. A very small amount of 1,4-dioxane was observed in IE1, likely due to the 1 molar % of ≥2EO oligomers of Structure (I). It is expected that C₁₄EO would provide the same low dioxane concentrations as it similarly has a single EO unit.

### Sulfated Surfactants

Table 3 provides dioxane measurements on the sulfated surfactants.

TABLE 3

| | | Dioxane (ppm) | | | | |
|---|---|---|---|---|---|---|
| | | | | Aqueous | | |
| | | Organic Phase | Organic Phase | Phase | | Relative to |
| | Sample | (GC at 110° C.) | (GC at 280° C.) | (LCMS) | Average^{b} | Solids^{c} |
| CE3 | SA3EO sulfate | 2.0 | 1471 | 0.48 | 1.24 | 15.3 |
| CE4 | ALEO1 sulfate | 0.9 | 259 | 0.51 | 0.71 | 9.5 |
| IE3 | C₁₂EO sulfate | <0.1^{a} | 0.58 | <0.1^{a} | <0.1^{a} | <1.6^{a} |

^{a}= Dioxane was not detected to a limit of 0.1 ppm
^{b}= Dioxane concentration was calculated from the average concentration in the aqueous phase and organic phase at 110° C.
^{c}= Dioxane concentration relative to solids was calculated based on the average concentration
^{d}= Calculated based on the limit of detection (0.1) ppm

Gas chromatography results of CE3 indicate the secondary alcohol 3EO sulfate contained 15.3 ppm of dioxane relative to solid and 2.0 ppm of dioxane in organic phase at 110° C. and indicates the problem of ≥2EO units can produce dioxane. Interestingly, when the inlet temperature was increased to 280° C., the dioxane content of secondary alcohol 3EO sulfate significantly increased to from 2 ppm to 1471 ppm. This result indicates that sulfated surfactants having ≥2 EO may develop observable dioxane at 110° C., but also that such surfactants may not stable at elevated temperatures of 280° C. which results in significant dioxane formation. Similarly to CE3, gas chromatography results of CE4 (i.e., the sulfated ALEO1) indicate the formation of unacceptable amounts of dioxane (i.e., >9 ppm) relative to the solids. Further, CE4 also exhibits a large generation of dioxane (259 ppm) at 280° C. suggesting that CE4 is not stable at high temperatures. IE3 demonstrates a low dioxane content due to the 95 mol % or greater of 1EO oligomers and 5 mol % or less of ≥2EO oligomers. Surprisingly, IE3 also demonstrates an extremely low dioxane content and falls below the limit of detection (LOD) of the GC and LC methods. Using the LOD as the basis, this indicates that the dioxane content for IE3 is <1.6 ppm, relative to solid, at 110° C. Interestingly, when the inlet temperature is increase to 280° C., the dioxane content of the IE3 material is still less than 1.0 ppm (0.58 ppm). This indicates C₁₂EO not only can prevent the dioxane formation in the sulfation process, but also has increased thermal stability, to resist dioxane formation, relative to the comparative examples.

What is claimed is:

1. A process, comprising the steps of:
contacting an olefin, an alcohol and a metallosilicate catalyst to form oligomers of an alcohol ethoxylate having structure (I) 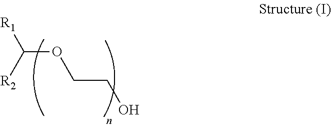
wherein R₁ is an alkyl, R₂ is an alkyl, and n has a value of 1 to 3; and
sulfating the oligomers of Structure (I) to form oligomers of Structure (II) 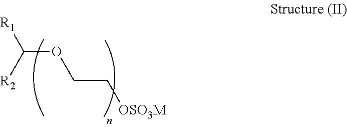
wherein R₁ is an alkyl, R₂ is selected from the group consisting of an alkyl group, M is selected from the group consisting of a proton, an ammonium cation, a metal cation, a nitrogen cation, a boron cation, a phosphorus cation, triethylamine, triethanolamine, monoethanolamine and combinations thereof, and n has a value of 1 to 3,
wherein 95 mol % or greater of the oligomers of Structure (II) have an n of 1 and 5 mol % or less of the oligomers of Structure (II) have an n of 2 or greater.

2. The method of claim 1, wherein the olefin is a C₈ to C₁₈ alpha olefin and the alcohol is monoethylene glycol.

3. The method of claim 1, wherein a sum of carbon atoms in R₁ and R₂ is from 7 to 17.

4. The method of claim 3, wherein a sum of carbon atoms in R₁ and R₂ is from 11 to 13.

5. The method of a claim 1, wherein the catalyst to make Structure (I) has a surface area of 680 m²/g as measured according to ASTM D4365-19.

6. The method of claim 1, wherein 98 mol % or greater of the oligomers of Structure (I) have an n of 1 and 2 mol % or less of the oligomers of Structure (I) have an n of 2 or greater.

7. A surfactant composition, comprising,
oligomers of a surfactant having structure (II) 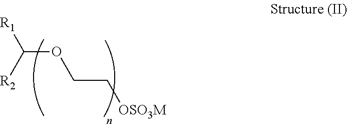
wherein R₁ is an alkyl, R₂ is an alkyl, M is selected from the group consisting of a proton, an ammonium cation, a metal cation, a nitrogen cation, a boron cation, a phosphorus cation, triethylamine, triethanolamine, monoethanolamine and combinations thereof, and n has a value of 1 to 3,
wherein 95 mol % or greater of the oligomers of Structure (II) in the composition have an n of 1 and 5 mol % or less of the oligomers of Structure (II) in the composition have an n of 2 or greater.

8. The surfactant composition of claim 7, wherein R₁ of Structure (II) is a C₁ to C₁₇ alkyl, R₂ is selected from the group consisting of a C₁ to C₁₆ alkyl, further wherein a sum of carbon atoms in R₁ and R₂ is from 7 to 17.

9. The surfactant composition of claim 7, wherein 98 mol % or greater of the oligomers of Structure (II) in the surfactant composition have an n of 1 and 2 mol % or less of the oligomers of Structure (II) in the surfactant composition have an n of 2 or greater.

10. The surfactant composition of claim 7, wherein a sum of carbon atoms in R₁ and R₂ is from 11 to 13.

11. A material comprising:
0.1 wt % to 99.9 wt % of the surfactant composition of claim 7.
